(19) United States
(12) Patent Application Publication
(10) Pub. No.: US 2023/0200377 A1
(43) Pub. Date: Jun. 29, 2023

(54) DEVICE FOR FREEZING AND TRANSFERRING GRAFT AND METHOD OF FREEZING AND TRANSFERRING GRAFT

(71) Applicant: TERUMO KABUSHIKI KAISHA, Tokyo (JP)

(72) Inventor: Fumiya OHASHI, Kanagawa (JP)

(73) Assignee: TERUMO KABUSHIKI KAISHA, Tokyo (JP)

(21) Appl. No.: 18/165,446

(22) Filed: Feb. 7, 2023

Related U.S. Application Data
(63) Continuation of application No. PCT/JP2021/035489, filed on Sep. 28, 2021.

(30) Foreign Application Priority Data
Sep. 28, 2020 (JP) 2020-162463

(51) Int. Cl. A01N 1/02

(52) U.S. Cl. CPC A01N 1/0268; A01N 1/0284; A01N 1/0221

(57) ABSTRACT

An object of the present invention is to provide a unit capable of efficiently and quickly detaching a graft from a device without causing damage or wrinkles. The above problem has been solved by a medical device that transfers a graft to a target site, the medical device including the graft and a support with the graft placed on one surface, in which the graft and the support are frozen in a state where the graft is placed, and the graft can be detached from the support and transferred to the target site by thawing the frozen graft placed on the support.

### CROSS-REFERENCES TO RELATED APPLICATIONS

This application is a continuation of International Application No. PCT/JP2021/035489 filed on Sep. 28, 20221, which claims priority to Japanese Patent Application No. 2020-162463 filed on Sep. 28, 2020, the entire content of both of which is incorporated herein by reference.

### TECHNOLOGICAL FIELD

The present invention generally relates to a medical device for freezing and transferring a graft to a target site and a method of freezing and transferring a graft to a target site.

### BACKGROUND DISCUSSION

In recent years, attempts have been made to transplant various cells for repair of damaged tissues and the like. For example, attempts have been made to use fetal cardiomyocytes, myoblast cells, mesenchymal stem cells, cardiac stem cells, ES cells, iPS cells, and the like for repair of myocardial tissue damaged by ischemic heart disease such as angina pectoris and myocardial infarction (Haraguchi et al., Stem Cells Transl Med. 2012 February; 1(2): 136-41).

As a part of such attempts, a cell structure formed using a scaffold and a sheet-shaped cell culture in which cells are formed in a sheet shape have been developed (Sawa et al., Surg Today. 2012 January; 42(2): 181-4).

For the application of the sheet-shaped cell culture to treatment, studies have been made on the use of a cultured epidermal sheet for skin damage caused by burns or the like, the use of a corneal epithelial sheet-shaped cell culture for corneal damage, the use of an oral mucosa sheet-shaped cell culture for endoscopic resection of esophageal cancer, and the like, and some of them have entered the stage of clinical application.

Tanaka et al., Surg Today. 2017 January; 47(1): 114-121 describes that a myoblast cell sheet can be used for healing pancreatic fistula and gastric perforation in a model animal. In recent years, endoscopic submucosal dissection (ESD) has attracted attention as a method for minimally invasive resection of an early tumor. This is to inject a local injection solution into the submucosa layer of the lesion, artificially cause edema, and then excise the raised mucosal lesion together with the submucosa layer. However, it has been reported that ESD particularly in the duodenum causes complications such as perforation with a probability of around 30% due to operability of the endoscope, thinness of the intestinal wall, and the like.

With regard to a surgical method for administering a sheet-shaped cell culture to a target site, endoscopic surgery is widely used as a minimally invasive surgical method for a human body, and various instruments for delivering and administering a sheet-shaped cell culture to a target site have been proposed.

For example, a conveyance and administration device described in Japanese Patent Application Publication No. 2009-511 (JP 2009-511 A) can convey a sheet-shaped therapeutic substance to an implantation site with a low degree of invasion to the human body by housing a sheet support member in a cylindrical shape in an outer cylinder.

A sheet pasting device described in Japanese Patent Application Publication No. 2016-187601 (JP 2016-187601 A) can detach a rod-like member disposed along a surface of a sheet support by moving the rod-like member along the surface of the sheet support to paste a sheet-like substance to a target site.

In a transport administration device described in Japanese Patent Application Publication No. 2008-173333 (JP 2008-173333 A), a sheet can be administered to a sheet support by a sheet attaching and detaching unit capable of applying a negative pressure for adsorbing and holding a sheet-like therapeutic substance and a positive pressure for detaching the sheet-like therapeutic substance from the sheet support.

A conveying device described in International Patent Application Publication No. 2014/069292 (WO 2014/069292 A) can attach a sheet-like therapeutic substance by ejecting a fluid from a plurality of vent holes provided on a surface of a head holding the sheet-like therapeutic substance.

### SUMMARY

The present inventor has worked on developing a unit for efficiently transferring the graft to a target site, and has discovered that problems and difficulties can arise when detaching a graft from a device without causing damage or wrinkles. The inventor here has discovered a simple configuration and reasonable medical device that is not so susceptible to such difficulties.

While conducting intensive research to solve the above problem, the present inventor has focused on the fact that a graft can be appropriately detached by freezing a support on which the graft is placed, delivering the support to the target site, and thawing the support. Then, the present inventor has found that it is possible to efficiently transfer a graft to a target site by using a medical device including a support capable of placing the graft on one surface and freezing the graft, and as a result of further research, has completed the device and method disclosed here, representing examples of the inventive developments disclosed here.

According to one aspect, the device disclosed here relates to the following.

[1] A medical device includes: a graft; and a support having opposite surface, with the graft being positioned on one of the surfaces of the support. The graft and the support are frozen in a state where the graft is positioned on the one surface of the support, and the frozen graft positioned on the one surface of the support is to be thawed to allow the graft to be detached from the support and transferred to the target site.

[2] The medical device according to [1], in which a frozen liquid is between the graft and the support.

[3] The medical device according to [2], in which the frozen liquid contains a frozen cryoprotective agent.

[4] The medical device according to any one of [1] to [3], in which the support includes a plurality of holes.

[5] The medical device according to any one of [1] to [4], in which the support contains a material having high thermal conductivity.

[6] According to another aspect, a medical device that transfers a graft to a target site includes a support on which the graft is to be positioned and frozen. The support is freezable together with the graft while the graft is positioned on the support and thereafter being subjected to thawing together with the graft to allow the graft to be detached from the support and transferred to the target site.

[7] According to another aspect, a method includes: positioning a graft on a surface of a support; freezing the graft and the support; transferring the frozen support on which the frozen graft is supported adjacent to the target site; thawing the frozen graft supported on the support; and detaching the thawed graft from the support and transferring the thawed graft to the target site.

[8] The method according to [7], in which the thawing is performed by warming only the support.

[9] The method according to [7] or [8], in which the thawing is performed by bringing the support on which the graft is supported into contact with the target site.

[10] The method according to any one of [7] to [9], in which the transferring of the graft to the target site is performed by inserting the support on which the graft is supported into a cylindrical body inserted intraperitoneally into a body cavity.

[11] The method according to any one of [7] to [10], in which the graft is intraperitoneally inserted into a body cavity and attached to the target site.

According to the device and method, the graft can be easily detached from the support and transferred to the target site. Furthermore, it is possible to fix the graft to the target site while maintaining a shape of the graft without causing damage or wrinkles. Moreover, freezing the support on which the graft is placed allows the graft to be reliably transferred to the target site even when the device is in an upward-facing, downward-facing, or sideward-facing state.

### BRIEF DESCRIPTION OF THE DRAWINGS

FIGS. 1A and 1B illustrate a device disclosed by way of example.

FIG. 2 is a conceptual or schematic diagram of one aspect of transferring a graft to a target site using the device.

### DETAILED DESCRIPTION

In the description below, the “graft” means a structure for transplantation into a living body, and particularly means a structure for transplantation containing living cells as a component. Preferably, the graft is a structure for transplantation that does not contain living cells and a structure other than a substance derived from living cells (for example, a scaffold or the like). Examples of the graft include, but are not limited to, a sheet-shaped cell culture, a spheroid, a cell aggregate, and the like, preferably a sheet-shaped cell culture or a spheroid, and more preferably a sheet-shaped cell culture.

In the description below, the “sheet-shaped cell culture” refers to a sheet-shaped culture in which cells are linked to each other. Cells may be linked to one another directly (including via cellular elements such as adhesion molecules) and/or via an intervening substance. The intervening substance is not particularly limited as long as it is a substance capable of at least physically (mechanically) linking cells to each other, and examples thereof include an extracellular matrix and the like. The intervening substance is preferably derived from a cell, in particular, derived from a cell constituting a cell culture. The cells are linked at least physically (mechanically), but may also be linked functionally, for example chemically or electrically. The sheet-shaped cell culture may be composed of one cell layer (monolayer) or composed of two or more cell layers (laminated (multilayer) body, for example, two layers, three layers, four layers, five layers, six layers, and the like). Furthermore, the sheet-shaped cell culture may have a three-dimensional structure having a thickness exceeding a thickness of one cell without the cells exhibiting a clear layer structure. For example, in a vertical cross-section of the sheet-shaped cell culture, cells may be present in a non-uniform arrangement (for example, in a mosaic manner) without being uniformly aligned in the horizontal direction.

The sheet-shaped cell culture preferably does not contain a scaffold (support). Scaffolds may be used in the technical field to attach cells on and/or within their surface and maintain the physical integrity of the sheet-shaped cell culture, for example, membranes made of polyvinylidene difluoride (PVDF) and the like are known, but the sheet-shaped cell culture described below can maintain its physical integrity without such a scaffold. Furthermore, the sheet-shaped cell culture described below is preferably composed only of substances derived from cells constituting the sheet-shaped cell culture, and does not contain other substances.

The cells constituting the sheet-shaped cell culture are not particularly limited as long as they can form the sheet-shaped cell culture, and include, for example, adherent cells (adherent cells). The adherent cells include, for example, adherent somatic cells (for example, cardiomyocytes, fibroblast cells, epithelial cells, endothelial cells, hepatocytes, pancreatic cells, renal cells, adrenal cells, periodontal ligament cells, gingival cells, periosteal cells, skin cells, synoviocytes, chondrocytes, and the like), and stem cells (for example, myoblasts, tissue stem cells such as cardiac stem cells, embryonic stem cells, pluripotent stem cells such as induced pluripotent stem (iPS) cells, mesenchymal stem cells, etc.). The somatic cells may be stem cells, particularly those differentiated from iPS cells (iPS cell-derived adherent cells). Non-limiting examples of cells that constitute the sheet-shaped cell culture include, for example, myoblasts (for example, myoblast cells, and the like), mesenchymal stem cells (for example, those derived from bone marrow, adipose tissue, peripheral blood, skin, hair roots, muscle tissue, endometrium, placenta, cord blood, and the like), cardiomyocytes, fibroblast cells, cardiac stem cells, embryonic stem cells, iPS cells, synoviocytes, chondrocytes, epithelial cells (for example, oral mucosal epithelial cells, retinal pigment epithelial cells, nasal mucosal epithelial cells, and the like), endothelial cells (for example, vascular endothelial cells, and the like), hepatocytes (for example, hepatocytes, and the like), pancreatic cells (for example, islet cells, and the like), renal cells, adrenal cells, periodontal ligament cells, gingival cells, periosteal cells, and skin cells. Non-limiting examples of the iPS cell-derived adherent cells include iPS cell-derived cardiomyocytes, fibroblast cells, epithelial cells, endothelial cells, hepatocytes, pancreatic cells, renal cells, adrenal cells, periodontal ligament cells, gingival cells, periosteum cells, skin cells, synovium cells, and chondrocytes.

In the following description, the “support” refers to a structure having a surface capable of supporting a graft while maintaining its shape. The support can be made of a material that can be frozen and warmed in a state where the graft is placed on one surface thereof and that can cope with a rapid temperature change. When the graft is placed on the support and frozen, liquid attached to the graft is frozen between the graft and the support to act as an adhesive. In a case where the support has a plurality of holes, liquid attached to the graft is frozen in a state of entering the plurality of holes, whereby the graft can be more firmly held by the support. From the viewpoint of heating and thawing the graft and detaching the graft from the support, the support is preferably made of a material having high thermal conductivity. Furthermore, it is also possible to place a material having lower thermal conductivity on a material having higher thermal conductivity and place the graft thereon in order to prevent rapid heat transfer to the graft. The thermal conductivity of the support is not particularly limited, but can be set to 0.15 to 360 [W/mK], preferably 10 to 360 [W/mK], and more preferably 50 to 360 [W/m K]. The graft can also be detached by natural thawing of the graft placed on the support without warming the support.

In one aspect, the support has flexibility and moderate softness, and can be curved with the graft supported and stored, such as along the inside of a tubular body, and deployed in a planar manner with its resiliency when exposed from the tubular body. A material of the support is not particularly limited, and various known materials can be used alone or in combination. Examples of such a material include a metal, a soft vinyl chloride resin, a polyurethane resin, a silicone rubber, a natural rubber, a synthetic rubber, a styrene-ethylene-butylene-styrene copolymer (SEBS), a styrene-ethylene-propylene-styrene copolymer (SEPS), an ethylene-vinyl acetate copolymer (EVA), a polyamide resin and a polyamide elastomer such as nylon, a polyester resin and a polyester elastomer such as polyethylene terephthalate (PET), an olefin-based resin such as polyethylene, a fluororubber, and a fluororesin, and one or a combination of two or more of these can be used.

The “plurality of holes” referenced in the description below refers to two or more through holes provided in the support, and a fluid is fed from a surface opposite to a surface supporting the graft of the support, and the fluid is discharged through the holes, whereby the graft can be detached from the support surface and attached. It is sufficient that the fluid can be discharged, and at least one hole may be provided in the support.

Furthermore, the “plurality of holes” preferably have a hole diameter that allows liquid attached to the graft to enter and be held by surface tension when the graft is placed on the support. As a result, the graft, the support, and the liquid entering the plurality of holes are frozen, so that the graft is firmly held by the support.

The plurality of holes are preferably discretely arranged on a surface of the support supporting the graft so that a detaching position and a holding position of the graft can be adjusted. For example, at least one of the plurality of holes may be provided near an edge of the support so that the graft can be detached from near the edge. The arrangement of the plurality of holes can be a matrix arrangement of rows x columns, for example, 2×2 to 40×40, 5×5 to 20×20, etc., a spacing between the holes can be, for example, 0.05 mm to 10 mm, 0.5 mm to 2 mm, and a diameter of the holes can be, for example, 0.05 mm to 10 mm, 0.3 to 0.6 mm, but is not limited thereto, and those skilled in the art can freely select according to a size of the graft, the degree of adhesion between the graft and the support, and the like. The matrix arrangement of the plurality of holes can be achieved by forming the structure of the support into a network structure.

In the description below, the “fluid” refers to a generic term for a gas and a liquid that can be extruded and detached without damaging a graft. The liquid is composed of at least one component, and the component is not particularly limited, but is composed of, for example, a liquid such as water, an aqueous solution, a non-aqueous solution, a suspension, or an emulsion. The component constituting the liquid is not particularly limited as long as it has little influence on the graft. In a case where the graft is a membrane made of a biological material, the component constituting the liquid is preferably a biocompatible component, that is, a component that does not cause an undesirable action such as an inflammatory reaction, an immune reaction, or an intoxication reaction on a biological tissue or a cell, or at least has a small action.

The “target site” referenced in the following description refers to a portion to which the graft is applied (attached). Examples of the target site include a site where a damage (wound) exists in a hollow organ, an opposite side of a lumen wall corresponding to a site where a damage exists, and the like. The “hollow organ” means an organ having a lumen housed in a body cavity, that is, an organ having a tubular or bag-like structure, and examples thereof include organs of a digestive tract system, a circulatory system, a urinary tract system, a respiratory system, and a female reproductive system. It is typically an organ of the digestive tract system. Furthermore, the “lumen wall” means an organ wall constituting a tube or a bag of a hollow organ. The lumen wall has an inner portion facing the lumen and an outer portion forming an outer surface of the organ. The term “one side of the lumen wall” means either the inner side or the outer side of the lumen wall, and the outer side with respect to the inner side or the inner side with respect to the outer side is referred to as “opposite side”. Furthermore, a region located just opposite the lumen wall with respect to a certain region on one side is referred to as a “corresponding opposite side”.

In a certain aspect, in luminal tissue having damage on at least one side of the luminal wall, tissue healing can be promoted by transplanting a sheet-shaped cell culture on an opposite side corresponding to a site where the damage is present. In particular, even damage formed by an operation such as ESD is effective, so that it is possible to prevent complications after these operations and improve the prognosis.

In the following description, the “liquid” is composed of at least one kind of component, and the component is not particularly limited, but is composed of, for example, a liquid such as water, an aqueous solution, a non-aqueous solution, a suspension, an emulsion, a culture solution, or a physiological buffer solution.

The component constituting the liquid is not particularly limited as long as it has little influence on the graft. In a case where the graft is made of a biological material, the component constituting the liquid is preferably a biocompatible component, that is, a component that does not cause an undesirable action such as an inflammatory reaction, an immune reaction, or an intoxication reaction on biological tissue or cells, or at least has a small action, from the viewpoint of biological stability and long-term storage possibility. Examples of the component include water, physiological saline, physiological buffer solution (for example, HBSS, PBS, EBSS, Hepes, sodium bicarbonate, and the like), medium (for example, DMEM, MEM, F12, DMEM/F12, DME, RPMI1640, MCDB, L15, SkBM, RITC80-7, IMDM, and the like), sugar solution (sucrose solution, Ficoll-paque (registered trademark) PLUS, and the like), sea water, serum-containing solution, lenographin (registered trademark) solution, metrizamide solution, meglumine solution, glycerin, ethylene glycol, ammonia, benzene, toluene, acetone, ethyl alcohol, benzol, oil, mineral oil, animal fat, vegetable oil, olive oil, colloid solution, liquid paraffin, turpentine oil, linseed oil, castor oil, and the like.

In a case where the graft is a sheet-shaped cell culture, the component constituting the liquid can stably preserve cells, preferably contains minimum oxygen, nutrients, and the like necessary for cell survival, and does not destroy the cells by osmotic pressure or the like, and examples thereof include, but are not limited to, physiological saline, physiological buffer solution (for example, HBSS, PBS, EBSS, Hepes, sodium bicarbonate, and the like), medium (for example, DMEM, MEM, F12, DMEM/F12, DME, RPMI1640, MCDB, L15, SkBM, RITC80-7, IMDM, and the like), sugar solution (sucrose solution, Ficoll-paque PLUS (registered trademark), etc.), and the like.

In the description below, the “liquid” can further include a cryoprotective agent used for freezing cells. The freeze operation of the graft may be performed after the graft is immersed in a culture solution, a physiological buffer solution, or the like, but may be performed after a treatment such as adding a cryoprotective agent for protecting cells from freeze/thaw operation to the culture solution or replacing the culture solution with a cryopreservation solution containing a cryoprotective agent is performed. The cryoprotective agent used for freezing the graft is not particularly limited as long as it exhibits a cryoprotective effect on cells, and examples thereof include dimethyl sulfoxide (DMSO), glycerol, ethylene glycol, propylene glycol, sericin, propanediol, dextran, polyvinyl pyrrolidone, polyvinyl alcohol, hydroxyethyl starch, chondroitin sulfate, polyethylene glycol, formamide, acetamide, adonitol, perseitol, raffinose, lactose, trehalose, sucrose, mannitol, and the like. The cryoprotective agent may be used alone or in combination of two or more.

The step of freezing the graft and the support in a state where the graft is placed can be performed by any known technique used for freezing the graft including cells and the like. Such techniques include, but are not limited to, subjecting the support on which the graft is placed to a freezing unit, e.g., a freezer, a deep freezer, a cold medium (for example, liquid nitrogen or the like), etc. The temperature of the freezing unit is not particularly limited as long as it is a temperature at which a part of the graft, preferably the entire graft, can be frozen, but is typically about 0° C. or less, preferably about −20° C. or less, more preferably about −40° C. or less, and still more preferably about −80° C. or less. Furthermore, a cooling rate in the freezing operation is not particularly limited as long as the survival rate and function of the cells after freeze-thawing are not significantly impaired, but is typically a cooling rate of about 1 hour to about 5 hours, preferably about 2 hours to about 4 hours, and particularly about 3 hours (slow freezing) from the start of cooling from 4° C. until reaching −80° C. Specifically, for example, cooling can be performed at a rate of about 0.46° C./min. Such a cooling rate can be achieved by subjecting the support on which the graft is placed to the freezing unit set to a desired temperature directly or by accommodating the support in a freezing treatment container. The freezing treatment container may have a function of controlling a lowering speed of the temperature in the container to a predetermined speed. As such a freezing treatment container, any known container, for example, BICELL® (Nihon Freezer Co., Ltd.) or the like can be used.

Hereinafter, a device according to a preferred embodiment will be described in detail with reference to the drawings.

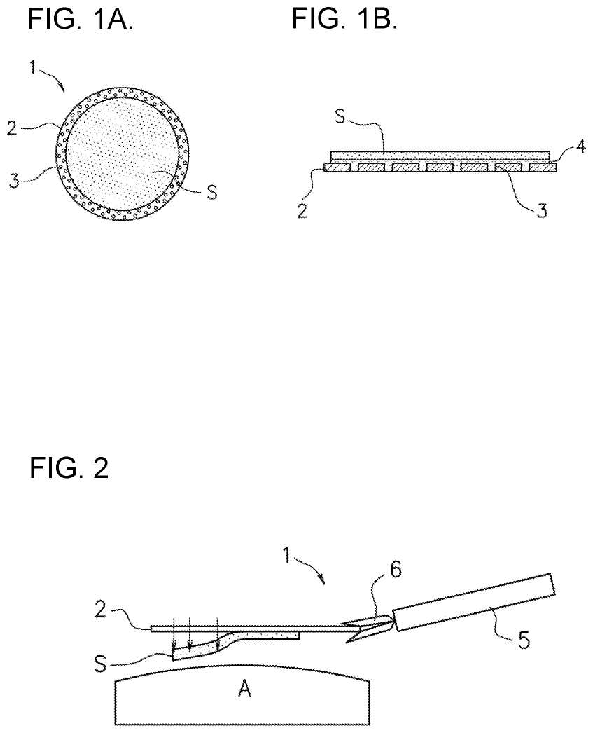

As illustrated in FIGS. 1 and 2, a device 1 according to a first embodiment includes a graft S and a support 2 with the graft S placed on one surface of the support 2, and the graft S and the support 2 are frozen in a state where the graft S is placed or located on the support 2. For example, the device 1 can be realized by taking out the graft S immersed in a liquid 4 (which may contain a cryoprotective agent), placing the graft S that has been removed from the liquid 4 on one surface of the support 2, and freezing the graft S, the liquid 4, and the support 2. That is, the liquid 4 attached to the graft S is frozen between the graft S and the support 2 to serve as an adhesive, and the graft S can be appropriately held on the support 2. Therefore, even in a state where the device 1 is directed upward, downward, or sideways, the graft S can be transferred to a target site A without being detached.

In one aspect, the support 2 may have a plurality of holes 3. As a result, the liquid 4 attached to the graft S enters the plurality of holes 3 (FIG. 1B), and the liquid 4 is frozen, whereby the graft S is more firmly held by the support 2. Furthermore, by releasing the fluid from some of the plurality of holes 3 (i.e., allowing the fluid to flow through the holes), it is possible to more reliably detach the graft S from the support 2 and fix the graft S to the target site A. In one aspect, the support 2 can be made of a material having high thermal conductivity (such as a metal filter). As a result, it is possible to indirectly transfer heat to the graft S and thaw the graft S only by heating the support 2, and it is possible to appropriately detach the graft S without directly thermally damaging the graft S. Furthermore, it is also possible to detach the graft placed on the support by natural thawing.

In one aspect, the support 2 is a plate-like body having flexibility and moderate softness, and can be curved in a state of supporting the graft S and inserted (stored) into a cylindrical body 5 or the like. It is also possible to configure the support such that a part of the outline of the support 2 is molded so as to have a convex shape (for example, in a circular shape), and a top portion of the convex shape portion is curved by being brought into contact with and being pressed against an opening portion of the cylindrical body 5 so that the support 2 can be easily pushed into the cylindrical body 5 or the like. In a case where the support 2 on which the graft S is placed is frozen, the graft S can be easily inserted into the cylindrical body 5 or the like by curving (rounding) or rolling and then freezing the graft S.

In one aspect, the support 2 can also be configured to be able to be developed into a planar manner (i.e., to naturally assume or take on a planar configuration) by virtue of its resiliency when pushed out (or pulled out) from the cylindrical body 5. For example, by using a port inserted intraperitoneally into a body cavity used in an endoscopic surgical operation or the like as the cylindrical body 5, the support 2 can be configured to be automatically developed in a planar shape when the support 2 is pushed out from the port and brought close to the target site A. The support 2 supporting the graft S is rolled so that the graft S is inside the rolled-up support 2, and the rolled-up support 2 with the graft S inside is inserted into the opening portion of the cylindrical body 5 from the outside of the body in a state where a peripheral edge portion of the support 2 is gripped by a forceps 6 or the like. In this way, the support 2 (and the graft S) can be appropriately transferred to the target site A without damaging the graft S.

As illustrated in FIG. 2, when using the device 1, the support 2 supporting the frozen graft S can be positioned proximate the target site A, and the graft S, liquid 4 and/or support 2 can be thawed by natural thawing of the graft S placed on the support 2 or by warming of the support 2. As a result, the adhesiveness of the liquid 4 serving as an adhesive at the time of freezing is weakened, and the graft S can be easily detached from the support 2 and transferred to the target site A. The warming can be performed by warming the support 2 on which the graft S is placed or by bringing the support 2 on which the graft S is placed into contact with the target site A. That is, the warming includes warming the graft S and/or the support 2.

The warming can be realized by bringing hot air, a warmed member, or the like into contact with the support 2 on which the graft S is placed, configuring the forceps 6 or the like for gripping the support 2 to be temperature-adjustable, configuring the support 2 itself to be temperature-adjustable, or the like. In a case where only the support 2 is warmed, heat can be indirectly transferred to the graft S (and the intervening liquid 4) to prevent direct thermal damage from being applied to the graft S. Configuring the support 2 itself to be temperature-adjustable may include configuring the support 2 itself to be cooling-adjustable. That is, the temperature of the support 2 can be lowered while the graft S is placed on the support 2, and the graft S and the support 2 can be frozen. In the case of natural thawing, since the graft S is not warmed, it can be thawed without causing thermal damage to the graft S.

The support 2 on which the graft S is placed may be warmed by, for example, bringing the support 2 on which the graft S is placed into contact with (attaching to) the target site A. That is, by bringing the surface of the support 2 on which the graft S is placed into contact with the target site A, the temperature of the target site A can be transferred to the graft S and/or the support 2 to be warmed. Thereby, the graft S can be easily detached from the support 2. Furthermore, in this case, the graft S can be brought into contact with the target site A while the shape of the graft S is more appropriately held, and can be detached from the support 2, that is, fixed to the target site A, so that the graft S can be transferred without causing damage or wrinkles.

In a case where the support 2 has the plurality of holes 3, the detaching of the graft S from the support 2 as described above can also be ensured by releasing the fluid from some of the holes of the plurality of holes 3. After a part of the thawed graft S (for example, a peripheral portion) is fixed (attached) to the target site A, the support 2 is moved, so that the other part of the thawed graft S can be detached from the support 2 while pulling the graft S. That is, by detaching the graft S while pulling the graft S, the graft S can be fixed to the target site A in a stretched state, so that wrinkles or the like hardly occur in the graft S.

In a case where the graft S is transplanted to the target site A such as an organ using the device 1 disclosed here, the support 2 supporting the frozen graft S can be directly delivered into an abdominal cavity (in a body cavity), or the graft S can be delivered intraperitoneally into the body cavity via a hollow port (the cylindrical body or tube 5) penetrating an abdominal wall used in endoscopic surgery or the like. In a case where the graft S is delivered intraperitoneally, the frozen graft S and support 2 are inserted through a hollow port (the cylindrical body or tube 5) and delivered into the body cavity. At this time, by bringing the support 2 into a rolled-up state as described above, it is also possible to further prevent the possibility that the graft S is damaged and deliver the graft S.

The support 2 can be moved in the port by gripping the support 2 with the forceps 6 or the like and advancing and retracting the forceps 6. By extending the support 2 from the tip of the hollow port, the support 2 can be positioned with respect to the target site A. Positioning of the support 2 to the target site A may be performed using another forceps (not illustrated) inserted into the abdominal cavity. After positioning the support 2, the graft S is naturally thawed, or the support 2 is warmed to detach the graft S, and the graft S is transplanted to the target site A. The warming of the support 2 can be realized by warming the support 2 on which the graft S is placed or by bringing a surface of the support 2 on which the graft S is placed into contact with the target site A.

The device 1 only needs to include at least the support 2 on which the graft S can be placed on one surface and frozen. Furthermore, as described above, the device 1 may include the frozen graft S and the support 2 on which the graft S is placed on one surface. Moreover, as described above, the device 1 may further include the forceps 6, another forceps (not illustrated), the cylindrical body 5, the temperature-adjustable member, and the like. The support 2 may have the plurality of holes 3. After fixing the graft S to the target site A, the support 2 can also be retrieved using a laparoscopic surgical excisional bag (such as INZII® (registered trademark)).

The use of the device 1 can involve, for example, the following steps.

(1) The graft S and the support 2 are frozen in a state where the graft S is placed on or located on the support 2.

(2) The frozen graft S and support 2 are brought close to the target site A.

(3) The graft S is thawed and detached from the support 2.

(4) The detached graft S is fixed to the target site A.

Thawing of the graft S can be realized by warming the support 2 on which the graft S is placed or bringing a surface of the support 2 on which the graft S is placed into contact with the target site A. Furthermore, it is also possible to detach the graft S placed on the support 2 by natural thawing. The graft S to which the liquid containing the cryoprotective agent is attached can also be frozen together with the support 2.

In a case where the graft S placed on the support 2 is naturally thawed or in a case where the support 2 on which the graft S is placed is warmed, the support 2 on which the graft S is placed may be brought close to the target site A in a state of being spatially separated from the target site A. At this time, the support 2 can be floated in the air (adjacent to the target site A) so as not to come into contact with the target site A. This makes it possible to maintain a state in which the graft S is not in contact with the target site A until the graft S is attached to the target site A even after the graft S is detached from the support 2. The detaching of the graft S from the support 2 and the attachment of the graft S to the target site A may occur separately or simultaneously.

Furthermore, a step of injecting a fluid from above the attached graft S may be added after the attachment of the graft S. As a result, the graft S can be more reliably adhered to the target site A. Also at this time, the graft S can be adhered to the target site A while the support 2 is not in contact with the target site A. Furthermore, a reinforcing layer for reinforcing the sheet-shaped object (graft) may be formed by injecting a gel and/or a polymer as a fluid.

As described above, according to the device 1 according to the first embodiment, the graft can be easily detached from the support and transferred to the target site. Furthermore, it is possible to fix the graft to the target site while maintaining a shape of the graft without causing damage or wrinkles. Moreover, freezing the support on which the graft is placed allows the graft to be reliably transferred to the target site even when the device is in an upward-facing, downward-facing, or sideward-facing state.

The detailed description above describes embodiments of a medical device for freezing and transferring a graft to a target site and a method of freezing and transferring a graft to a target site representing examples of the medical device and method disclosed here. The invention is not limited, however, to the precise embodiments and variations described. Various changes, modifications and equivalents can be effected by one skilled in the art without departing from the spirit and scope of the invention as defined in the accompanying claims. It is expressly intended that all such changes, modifications and equivalents which fall within the scope of the claims are embraced by the claims.

### REFERENCE SIGNS LIST

1 Device
2 Support
3 Plurality of holes
4 Liquid
5 Cylindrical body
6 Forceps
S Graft
A Target site

What is claimed is:

1. A medical device used to effect transfer to a target site, the medical device comprising:
a graft; and
a support possessing opposite surfaces, the graft being in contact with and supported on one of the surfaces of the support; and
the graft and the support being frozen while the graft is in contact with and supported on the one surface of the support, and the graft being detachable from the one surface of the support and transferrable to the target site after thawing the graft.

2. The medical device according to claim 1, further comprising a frozen liquid between the graft and the support.

3. The medical device according to claim 2, wherein the frozen liquid includes a frozen cryoprotective agent.

4. The medical device according to claim 1, wherein the support includes a plurality of through holes.

5. The medical device according to claim 1, wherein the support contains a material having high thermal conductivity.

6. The medical device according to claim 1, wherein the support contains a material having a thermal conductivity of 0.15 to 360 [W/m K].

7. The medical device according to claim 7, wherein the support is an annular-shaped support surrounding an open central area.

8. The medical device according to claim 1, further comprising a frozen liquid between the graft and the support, the frozen liquid including a frozen cryoprotective agent, the support including a plurality of through holes and being an annular-shaped support surrounding an open central area.

9. A medical device used to transfer a graft to a target site, the medical device comprising a support on which the graft is positionable, the support being freezable together with the graft while the graft is positioned on the support and thereafter being subjected to thawing together with the graft to allow the graft to be detached from the support and transferred to the target site.

10. The medical device according to claim 9, wherein the support includes a plurality of through holes.

11. The medical device according to claim 9, wherein the support contains a material having high thermal conductivity.

12. The medical device according to claim 9, wherein the support contains a material having a thermal conductivity of 50 to 360 [W/m K].

13. The medical device according to claim 9, wherein the support is an annular-shaped support surrounding an open central area.

14. The medical device according to claim 9, wherein the support is annular in shape and surrounds an open central area, the support including plural spaced-apart through holes.

15. The medical device according to claim 14, wherein the support contains a material having a thermal conductivity of 50 to 360 [W/m K].

16. A method of transferring a graft to a target site, the method comprising:
positioning the graft on a support, the support possessing opposite surfaces, the positioning of the graft on the support including positioning the graft so that the graft is supported on one of the surfaces of the support;
freezing the graft and the support while the graft is supported on the one surface of the support;
transferring the frozen support on which the frozen graft is supported adjacent to the target site;
thawing the frozen graft positioned on the support;
detaching the graft that has been thawed from the one surface of the support; and
transferring the graft that has been detached from the one surface of the support to the target site.

17. The method according to claim 16, wherein the thawing of the frozen graft is performed by warming only the support.

18. The method according to claim 16, wherein the thawing of the frozen graft is performed by bringing the frozen support on which the frozen graft is positioned into contact with the target site.

19. The method according to claim 16, wherein the transferring of the graft to the target site is performed by inserting the support on which the graft is positioned into a cylindrical body inserted intraperitoneally into a body cavity.

20. The method according to claim 16, further comprising intraperitoneally inserting the graft into a body cavity and attaching the graft that has been intraperitoneally inserted into the body cavity to the target site.
